(19) United States
(12) Patent Application Publication
(10) Pub. No.: US 2017/0326118 A1
(43) Pub. Date: Nov. 16, 2017

(54) Drug treatment of tumors wherein hedgehog/smoothened signaling is utilized for inhibition of apoptosis of tumor cells

(71) Applicant: Sinan Tas, Bor (TR)

(72) Inventors: Sinan Tas, Bor (TR); Oktay Avci, Izmir (TR)

(21) Appl. No.: 15/530,513

(22) Filed: Jan. 23, 2017

Related U.S. Application Data
(63) Continuation-in-part of application No. 12/930,677, filed on Jan. 13, 2011.
Application No. 12/930,677 is a continuation of application No. 10/682,584, filed on Oct. 9, 2003, now Pat. No. 7,893,078.
Application No. 10/682,584 is a continuation-in-part of application No. PCT/TR02/00017, filed on Apr. 19, 2002.
Application No. PCT/TR02/00017 is a continuation-in-part of application No. PCT/TR01/00027, filed on Jul. 2, 2001.

(51) Int. Cl. A61K 31/4355; A61K 31/44; A61K 8/63; A61K 31/58; A61Q 19/02; A61K 45/06

(52) U.S. Cl. CPC A61K 31/4355; A61Q 19/02; A61K 45/06; A61K 8/63; A61K 31/58; A61K 31/44; A61K 2300/00

(57) ABSTRACT

This invention concerns use of cyclopamine or another selective inhibitor of hedgehog/smoothened signaling in vivo on basal cell carcinomas and other tumors wherein hedgehog/smoothened signalling is utilized for inhibition of differentiation and for inhibition of apoptosis of tumor cells to achieve differentiation and apoptotic death and removal of the tumor cells while preserving normal tissue cells and functions. Causation of apoptosis is by a non-genotoxic mechanism and thus unlike in the radiation therapy and most of the currently used cancer treatments which act by causing DNA-damage.

### CROSS REFERENCE

This application is a continuation-in-part of U.S. application Ser. No. 12/930,677, filed on 13 Jan. 2011 which is a continuation of U.S. application Ser. No. 10/682,584, filed on 9 Oct. 2003 which is a continuation-in-part of PCT/TR01/00027, filed on 2 Jul. 2001 designating the United States, and a continuation-in-part of PCT/TR02/00017, filed on 19 Apr. 2001 designating the United States. U.S. application Ser. No. 12/930,677, U.S. application Ser. No. 10/682,584, PCT/TR01/00027 and PCT/TR02/00017 are incorporated herein by reference in their entirety.

### BACKGROUND OF THE INVENTION

Basal cell carcinoma (BCC) is a common epithelial tumor. Its incidence increases with increasing age. Current treatments for BCC's include the surgical excision of the tumor together with a margin of normal tissue and, when surgery is not feasible or desirable, destruction of the tumor cells by ionizing radiation or other means. Although scarring and disfigurement are potential side effects, surgical excisions that do not leave neoplastic cells behind can provide cure. Radiation therapy acts by causing irreparably high quantity of DNA-damage which, in turn, triggers apoptotic death of the tumor cells. This mode of action of radiation-therapy, i.e. apoptosis secondary to DNA-damage, is similar to those of many chemotherapeutic agents that are currently used in the treatment of cancers. However, both radiation therapy and the cytotoxic cancer chemotherapeutics are capable of causing DNA-damage in the normal cells of patients in addition to the tumor cells. As a result, their effectivity and usefulness in cancer therapy are seriously limited. A further dilemma with the use of radiation and genotoxic cancer chemotherapeutics is the disturbing fact that, even when cure of the primary tumor is achieved, patients have markedly increased risk of developing new cancers because of the DNA-damage and the resulting mutations they have undergone during the treatment of primary tumor. Induction of apoptosis selectively in tumor cells by non-genotoxic means would therefore be most desirable in the field of cancer therapy.

BCC's frequently show inactivating mutations of the gene patched which encodes a transmembrane protein acting as a receptor for the hedgehog proteins identified first by their effect on the patterning of tissues during development. When not liganded by hedgehog, the patched protein acts to inhibit intracellular signal transduction by another transmembrane protein, smoothened. Binding of hedgehog to the patched causes relieving of this inhibition. Intracellular signal transduction by the relieved smoothened then initiates a series of cellular events resulting ultimately in alterations of the expressions of the hedgehog target genes and of cellular behaviour. General features of this hedgehog/smoothened pathway of signal transduction, first identified in Drosophila, are conserved in diverse living organisms from Drosophila to Human. However, the pathway gets more complex in more advanced organisms (e.g. presence in human of more than one genes that display significant similarity to the single patched gene of Drosophila). Inactivating mutations of the patched have been found to cause constitutive (ligand-free) signalling through the hedgehog/smoothened pathway. The hedgehog/smoothened pathway overactivity, resulting from mutations of the patched and/or further downstream pathway elements, is found in all BCC's. The nevoid basal cell carcinoma syndrome (NBCCS) results from patched haploinsufficiency. Patients with the NBCCS, because of an already mutant patched in all cells, develop multiple BCC's as they grow older. Hedgehog/smoothened signalling is known to be employed for normal functions in several normal tissues and for the maintenance of normal epithelial stem cells (Zhang Y et al (2001) Nature 410:599-604).

Cyclopamine, a steroid alkaloid, has the chemical formula shown below.

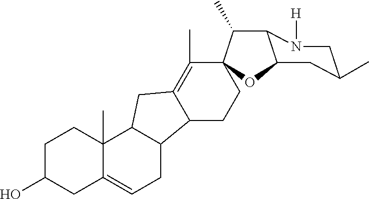

It is found naturally in the lily Veratrum californicum and can be obtained by purification from this and other sources. Inhibition of the hedgehog/smoothened pathway by cyclopamine has been found in chicken embryos and in cultured cells of mice. Cyclopamine has been found to inhibit the differentiation of neuronal precursor cells in developing brain (Incardona J P et al (1998) Development 125:3553-3562; Cooper M K et al (1998) Science 280:1603-1607). Studies with other differentiating cell types have also reported an inhibitory action of cyclopamine on cellular differentiation. Differentiation of bone marrow cells to erythroid cells (Detmer K. et al (2000) Dev. Biol. 222:242) and the differentiation of urogenital sinus to prostate (Berman D M et al (2000) J. Urol. 163:204) have been found to be inhibited by cyclopamine. Inhibition of hedgehog/smoothened signalling by cyclopamine has been reported to exert no significant effect on the viability of cells (Taipale J. et al (2000) Nature 406; 1005-1009).

The Prior Art Concerning Hedgehog/Smoothened Signaling and Molecules that Provide its Selective Inhibition

Described first in a publication of the results of a systematic screen of the genes that affect pattern formation during embryo development (Nüsslein-Volhard C et al, Nature 1980; 287:795-801), the hedgehog gene and the molecular signaling initiated by its product have been found to be largely conserved in species from drosophila to human. Hedgehog gene encodes for a secreted processed polypeptide (abbreviated here as Hh). Binding of Hh to a transmembrane protein, Patched, on a receiving cell initiates a molecular signaling transduced in the cell by another transmembrane protein, Smoothened (abbreviated here as Smo). When not liganded by Hh, Patched inhibits the signaling activity of Smo and the binding of Hh to Patched relieves the inhibition of Smo by Patched. The signaling by the relieved Smo has been determined to have a single end point in the cell, the Ci/Gli transcription factors that recognize a consensus sequence in the Hh target genes and affect their transcription (Method N et al, Development 2001; 128:733-742). The Smoothened protein has been determined to be essential for the signaling initiated by Hh in diverse species (Struhl G et al, Development 1997; 124:2155-2165; Wang Q T et al, Development 2000; 127:3131-3139; Zhang X M et al, Cell 2001; 105:781-792).

Besides the genetic means targeting Hh or Smo, several compounds have been purpose made for selective inhibition of Hh/Smo signaling in animals. Affinity-purified and monoclonal function-blocking anti-Hh antibodies have been made and shown to provide selective inhibition of Hh/Smo signaling in the administered embryos by multiple criteria (e.g. Ericson J et al, Cell 1996; 87:661-673). The brain in the vertebrate embryos that has loss of Hh expression shows inhibition of differentiation of various neural cells that are normally induced by Hh and the animals show consequent brain malformations that include a fusion of the developing eyes, called cyclopia (Krauss S et al, Cell 1993; 75:1431-1444). Causation of such brain malformations and cyclopia and deaths of fetuses and mothers in the animals administered with the teratogenic Veratrum alkaloids cyclopamine or jervine had been determined in various vertebrate species (Keeler R F, Proceedings Of The Society For Experimental Biology and Medicine 1975; 149:302-306; Omnell M L et al, Teratology 1990; 42:105-119). Incardona I et al (Development 1998; 125:3553-3562) and Cooper M K et al (Science 1998; 280:1603-1607), using methods like in the earlier investigations of Ericson et al (ibid), described that exposure of developing chicken embryos to cyclopamine or jervine caused these brain malformations and cyclopia due to a direct and selective inhibition of Hh/Smo signaling in the animals. Administration of cyclopamine or jervine to the developing embryos was found to cause a phenocopy of a Hh loss-of-function mutation and several further test results showing a direct and selective inhibition of Hh/Smo signaling in the animals by these compounds were also described (Incardona et al, ibid; Cooper et al, ibid).

Automatable in vitro assays with a Gli recognition sequence-driven reporter have also been described and provide quantitative data about the inhibition of Hh/Smo signaling by candidate compounds rapidly (e.g. Sasaki H et al, Development 1997; 124:1313-1322). Using patched −/− cells in such an assay, Taipale J et al (Nature 2000; 406:1005-1009) described that cyclopamine inhibits Hh/Smo signaling downstream of Patched, at the level of Smo, and described a derivative of it that was found to be more potent in the same assay. Molecules of interest determined to inhibit Hh/Smo signaling in such in vitro screens can then be tested in an available animal model for suitability for selective inhibition of Hh/Smo signaling in animals. Gaffield W et al (Cellular and Molecular Biology 1999; 45:579-588) described results of such animal testing and selective inhibition of Hh/Smo signaling in the administered chicken embryos by cyclopamine and enhancement of the inhibitory activity by conversion of cyclopamine to its 4-ene-3-one derivative.

Methods employing developing chicken and other embryos as convenient tools have been widely used for determining whether or not a molecule of interest can be used for selective inhibition of Hh/Smo signaling in animals. Stenkamp D L et al (Developmental Biology 2000; 220:238-252) and Nasevicius A et al (Nature Genetics 2000; 26:216-220) have described that developing zebrafish provide a particularly suitable model due to the ease of observation of the effects of administered molecules and known Hh loss-of-function mutants. They have described purpose made new molecules for selective inhibition of Hh/Smo signaling and causation of such inhibition in the administered animals by multiple criteria, including the phenocopying of a loss-of-function mutation of Hh and selective inhibition of differentiation of various cell types in vivo that are normally induced by Hh (Stenkamp et al, ibid; Nasevicius et al, ibid). Treier M et al (Development 2001; 128:377-386) described use of a macromolecule (HIP) that selectively bound to Hh for selective inhibition of Hh/Smo signaling in vivo.

Hh and other proteins that take part in Hh/Smo signaling have been found to be expressed in adults of various species that have been investigated, including in human, throughout different tissues and organs (Hahn H et al, Journal of Biological Chemistry 1996; 271:12125-12128; Takabatake T et al, FEBS Letters 1997; 401:485-499; Traiffort E et al, European Journal of Neuroscience 1999; 11:3199-3214; Koebernick K et al, Mechanisms of Development 2001; 100:303-308).

Hh/Smo signaling has been described to be required for numerous normal functions in adults. Hair follicle epithelial cells that show continuity with the epidermal basal layer cells were found to show Hh/Smo signaling in adults and the hair cycle was found to be affected by Hh/Smo signaling (Sato N et al, Journal of Clinical Investigation 1999; 104:855-864). Hh/Smo signaling has been described to be required for normal stem cell functions in adults of various species (Zhang Y et al, Nature 2001; 410:599-604; Van der Eerden B C et al, Journal of Bone and Mineral Research 2000; 15:1045-1055; Detmer K et al, Blood Cells Molecules and Diseases 2000; 26:360-372). Detmer et al, ibid, described that formation of differentiated blood cells from CD34+ stem cells of adult human bone marrow is stimulated by Hh and that treatment of the cells in culture with cyclopamine blocked this effect.

### The Prior Art Concerning Tumorigenesis and Treatment of Tumor Bearing Patients

Tumorigenesis is found to be significantly associated with aging in human and in other investigated species. Frequencies of tumors of various organs increase with increasing age and with exposure to agents that cause damage to the genetic material. Investigations of experimental animals administered with varying amounts of such agents have shown serious limits of repair of such damage. Mutations and epigenetic changes that increase with aging in somatic cells under ordinary conditions are found to be further increased by such exposures and found to increase the probability of tumorigenesis. Subsequent investigations have revealed the particular genes whose mutations or epigenetic changes increased the probability of tumorigenesis and shown that childhood tumors are often seen in children born with mutations of the revealed genes and/or with mutations that predispose to new mutations. They have also shown that tumorigenesis is a multistep process that involves occurrences of mutations and epigenetic changes of multiple genes in the same cell.

Patients diagnosed to have a tumor are commonly treated by its surgical excision. When removal of a tumor by surgery is not feasible due to its site or stage or not preferable (e.g. a mutilating surgery), radiotherapy and/or chemotherapy have in general been used. Radiotherapy attempts to get rid of the tumor by delivery of radiation to the tumor cells. Its effectiveness is limited by the radiation harm to the normal cells and functions of patient. Many tumors are found to be unresponsive to radiation at doses life-threatening to the patient. Various drug treatments have been used for tumor patients not feasible to be saved by surgery and various effects have been described by uses of different drug molecules. Uses that provide inhibition of proliferation of tumor cells (e.g. by inhibition of nucleotide synthesis, of DNA synthesis or of other steps of proliferation) or cell death by necrosis or by apoptosis have been known besides other interventions. Responses to the drug treatments practiced in prior art are in general known to be affected by the histopathological class or type and organ of origin of a tumor. Harming of the normal cells and functions of patients by a drug administration is again a leading cause of therapeutic failure. Most of the drug treatment candidates contemplated from effects on tumor cells in vitro or in mice are found to be unsuited for treatment of tumor bearing human due to prohibitive effects on the normal cells and functions (Takimoto C H, Clinical Cancer Research 2001; 7:229-230).

Normal stem cell functions have been determined to be essential for survival of every person. Findings with the people accidentally exposed to varying doses of radiation as well as the experience with tumor patients have shown that a critical decrease of normal stem cell functions even in a single organ proves fatal. Myelosuppression refers to normal bone marrow functions. Normal hematopoietic stem cells and progenitors in bone marrow give rise to the normal blood cells including those essential for normal immune functions and defense against microorganisms. Blood cell collection and transfusion technologies have been relatively advanced to help to keep alive the people who experienced a decrease of them. Densow D et al (Stem Cells 1997; 15-Supplement 2:287-297) reviewing the findings with the people who received radiation due to nuclear accidents concluded that hematopoietic stem cell transplantation to these victims could help to save them when the subject did not have a major involvement of the normal functions of other organs. Reduction of the of the normal stem cell functions in skin was found to be particularly critical and no patient subjected to hematopoietic stem cell transplantation was found to survive when he or she had significant part of the skin involved (Densow et al, ibid; Pellegrini G et al, Transplantation 1999; 68:868-879 described likewise results with patients who had losses of normal stem cell functions only in skin). Singhal S et al (Bone Marrow Transplantation 2000; 26:489-496) reported that patients having hematopoietic system tumoral diseases could be saved from the death due to radiation and/or drug administrations when they are transplanted with normal hematopoietic stem cells from HLA-identical sibling donors. In accord with the earlier findings with other cancer patients they determined that normal CD34+ hematopoietic stem cells must be provided to the patients in numbers above a critical level to avoid lethal outcome. Adequacy of the normal stem cell functions was found to independently predict both the overall survival and treatment-related mortality of tumor patients (Singhal et al, ibid). Lowering of normal stem cell—progenitor cell functions below a margin is found to preclude a beneficial therapeutic result in tumor patients and the majority of drug treatment candidates are found to fail in treatment of tumor bearing human particularly for that reason (Takimoto, ibid).

Studies of tumor cells from patients having tumors of various organs have revealed that a subset of the tumors show Hh/Smo signaling overactivity (Fujita E et al, Biochemical and Biophysical Research Communications 1997; 238:658-664; Reifenberger J et al, Cancer Research 1998; 58:1798-1803 and the references therein). Quantitative analyses showed markedly greater Hh/Smo signaling activity in tumor cells than in the normal cells in the same patients (on average about 7× or greater in the case of basal cell carcinomas; Tojo M et al, Pathology International 1999; 49:687-694).

Predisposition to occurrences of some tumors by activation of Hh/Smo signaling was suggested also by the findings that nevoid basal cell carcinoma syndrome patients are born with a mutant patched allele in all cells to cause increase of Hh/Smo signaling activity due to the patched haploinsufficiency and that these patched+/− subjects develop basal cell carcinomas and certain other tumors as they grow older (Kimonis V E et al, American Journal of Medical Genetics 1997; 69:299-308). Animals engineered to have patched haploinsufficiency in all cells have also been found to show increased probability of occurrences of tumors of some organs as they grow older (Goodrich L V et al, Science 1997; 277:1109-1113; Aszterbaum M et al, Nature Medicine 1999; 5:1285-1291). Goodrich et al, ibid, reported that medulloblastomas were observed in about 8% of patched+/− mice at 5 weeks of age and in about 30% of them at 12 to 25 weeks of age. Aszterbaum et al, ibid, reported increased occurrences of skin tumors in patched+/− mice in comparison to wild-type control animals with aging and exposure to agents that cause damage to the genetic material. With ultraviolet irradiation of skin, 3% of the 3-8 months old patched+/− mice were found to show tumors of skin and 40% of the patched+/− mice older than 9 months were found to have skin tumors. Such irradiation is known to cause damage to the genetic material and increased probability of occurrences of mutations and epigenetic changes throughout the genome.

Besides the loss-of-function mutations of patched, certain gain-of-function mutations of smo have also been described to cause activation of Hh/Smo signaling and found in some tumors (Xie J et al, Nature 1998; 391:90-92; Reifenberger et al, ibid). In accord with the above mentioned findings with subjects born with a Hh/Smo signaling activating mutation in all cells and found to develop tumors from a very small proportion of the cells with aging and exposure to mutagens, Xie et al, ibid, also reported insufficiency of a constitutive activation of Hh/Smo signaling for neoplastic transformation. In an in vitro assay of neoplastic transformation using known oncogenic viral gene controls, they reported that no transformed foci were observed in cells transfected with a gain-of-function mutant smo alone that caused constitutive activation of Hh/Smo signaling.

Aszterbaum et al, ibid, reported that tumor cells rendered devoid of Hh/Smo signaling showed slowing of proliferation during a period of 10 months of observation in culture. Taipale J et al (Nature 2000; 406:1005-1009) also reported a slowing of proliferation of other transformed cells that were rendered devoid of Hh/Smo signaling by treatment with a derivative of cyclopamine.

### Principles of Drug Therapy Established in the Prior Art

Extensive experience with patients given various drug treatments has shown that whereas drug treatments of symptoms may help patients in the absence of a solution otherwise, determination of the critical upstream events of pathogenesis that lead to the occurrence of a disease is often a precondition of development of a new drug treatment that is effective and safe for the patients and such a treatment can put end to multiple symptoms simultaneously. A further principle of drug treatment that has also been established in the art is that once a pathological process upstream and critical in the occurrence of a disease is determined, a pharmaceutically active compound that selectively intervenes with it without harming the patient through an effect or effects on the innumerable physiological processes in the patient must be used for a new drug treatment based on that determination.

A well-known example illustrative about these principles has been the drug treatments of peptic ulcer patients practiced prior to the determination that an infection by Helicobacter pylori is a critical upstream event in the pathogenesis of that disease. The previous drug treatments that attempted to decrease the gastric acidity to help to heal the ulcers and to alleviate gastric pain were poorly effective and were made mostly unneeded with the introduction of drug treatments that got rid of the H. pylori infection and ulcers. Whereas the nature of the target in that disease (a pathological process caused by a bacterium that is easy to selectively target in human body) has facilitated development of a safe and effective drug treatment of peptic ulcer disease, the basic principle of selectively intervening with an identified pathological process has been repeatedly confirmed as a precondition of being able to avoid the side effects due to the drug effects on unintended events in patients as reviewed and described in the scientific journal articles about the drug treatments of various diseases excerpted below.

Delyani J A (Kidney International 2000; 57:1408-1411) reviewed treatment of the aldosterone mediated cardiovascular disease as follows. “ . . . aldosterone . . . can mediate edema”. “ . . . elevated levels . . . result in interstitial cardiac fibrosis”. “The limited utility of spironolactone owing to the . . . side effects has been especially frustrating given the . . . role of aldosterone in cardiovascular disease. To obviate these limitations, eplerenone is . . . developed . . . . Eplerenone is a competitive antagonist . . . with . . . excellent selectivity for the mineralocorticoid receptor”. Its “affinity is approximately 10- to 20-fold less than spironolactone for the aldosterone receptor . . . . However, unlike spironolacone, eplerenone has little affinity for other steroid receptors . . . there are no steroid-related adverse effects . . . phase I trials indicated . . . a good safety profile . . . effective in hypertension as well as heart failure”.

Weldon M J et al (Gut 1994; 35:867-871) reviewed treatment of inflammatory bowel disease as follows. “Greater understanding of inflammatory bowel disease, and . . . of the central role of activated T cells, has prompted a search for drugs”. “The goal is to provide more effective and less toxic therapy by developing treatment targeted to specific . . . effector mechanisms”. “More selective targeting of activated T cells is therefore needed. Since activated T cells in inflammatory bowel disease . . . express αIL-2r whereas . . . resting T cells do not, antibodies to this receptor would provide such selectivity”.

Ellis C N et al (New England Journal of Medicine 2001; 345:248-255) described a new drug treatment of psoriasis on the basis of the knowledge in prior art about the occurrence of psoriasis lesions as follows. “Psoriatic plaques are characterized by infiltration with CD45RO+ memory effector T lymphocytes”. “ . . . LFA-3-CD2 signal plays an important part in the activation of T lymphocytes”. “ . . . CD45RO+ T lymphocyte subgroups . . . contain the clonal precursors driving the pathogenic process”. “Alefacept selectively targets CD45RO+ memory effector T lymphocytes”. “ . . . alefacept . . . was designed to prevent the interaction between LFA-3 and CD2”. “ . . . patients receiving alefacept had a greater decrease in the psoriasis area-and-severity index than those receiving placebo”.

Timermans PBWM (Hypertension Research 1999; 22:147-153) reviewed treatment of angiotensin II receptor type 1 mediated hypertensive disease as follows. “Activation of RAAS is critically involved in the development and maintenance of hypertension and congestive heart failure . . . Ang II . . . is the primary mediator of the RAAS”. “ . . . selective . . . Ang II type 1 (AT1) receptor antagonists provided . . . benefits . . . avoid the nonspecificity of the Ang I converting enzyme . . . inhibitors”. “ . . . all of the known actions of Ang II could be blocked by losartan, emphasizing the major role of the AT1 . . . in the patho(physiological) actions of this hormone . . . it also clearly explains why most of the pharmaceutical effort has been focused on developing . . . AT1 . . . selective antagonists”.

Culman J (Experimental Physiology 2000; 85:757-767) reviewed uses of purpose-made antisense oligonucleotide compounds in drug treatment as follows. “ . . . classical pharmacologic approaches . . . are often based on the inhibition of biologically active proteins”. “Binding of antisense oligonucleotides to the complementary . . . sequence . . . results in a selective inhibition of transcription or translation . . . . This . . . represents a promising basis for . . . therapies”. “ . . . an important advantage of antisense strategy is . . . the ability to selectively inhibit the expression of biologically active proteins where . . . agents are not available or show limited selectivity”.

Pelaia G et al (Allergy 2000; 55 (Supplement 61):60-66) reviewed drug treatment of asthma as follows. “ . . . adenosine induces bronchoconstriction via stimulation of A1-receptors”. “Respirable antisense oligonucleotides . . . have been designed which hybridize to A1-receptor . . . thereby . . . selectively reducing A1-receptor number”. Reviewing the knowledge about the pathogenesis of asthma, they added “These new therapeutic approaches have the advantage . . . of being more specifically targeted on the pathogenetic events”. “ . . . all sharing a common basic principle; that is, to develop drugs more directly targeted on the pathophysiology of the disease”.

These descriptions of the drug treatments of patients having various diseases in scientific publications by independent scientists all emphasize the aforementioned same basic medical principles that have been established in the art and show also their rationale with examples.

### SUMMARY OF THE INVENTION

This invention concerns the use of cyclopamine in vivo on basal cell carcinomas (BCC's) to achieve therapeutic effect by causing differentiation of the tumor cells and, at the same time, apoptotic death and removal of these tumor cells while preserving the normal tissue cells, including the undifferentiated cells of the normal epidermal basal layer and hair follicles. Causation of apoptosis by cyclopamine is by a non-genotoxic mechanism and thus unlike the radiation therapy and most of the currently used cancer chemotherapeutics which act by causing DNA-damage. These novel effects, previously unachieved by a cancer chemotherapeutic, make the use of cyclopamine highly desirable in cancer therapy, in the treatment of BCC's and other tumors that use the hedgehog/smoothened signal transduction pathway for proliferation and prevention of apoptosis.

In one aspect, the present invention is directed to the use of cyclopamine or a pharmaceutically acceptable salt or a derivative of cyclopamine in the topical treatment of basal cell carcinomas, particularly for the manufacture of a pharmaceutical compound for use in the topical treatment of basal cell carcinomas.

In a further aspect, the invention is directed to the use of cyclopamine or a pharmaceutically acceptable salt of cyclopamine or a derivative thereof in the treatment of basal cell carcinomas by non-topical means, including by intratumoral injections, or for the manufacture of a pharmaceutical compound for use in such a treatment.

In a further aspect, the invention is directed to the use of cyclopamine or a pharmaceutically acceptable salt of cyclopamine or a derivative of cyclopamine in the treatment of tumors that use the hedgehog/smoothened signal transduction pathway for proliferation and/or for the prevention of apoptosis or cellular differentiation, or for the manufacture of a pharmaceutical compound for use in such treatment. The described new drug treatment exemplified by use of a known selective inhibitor of Hh/Smo signalling, cyclopamine, is for treatment of patients having a tumor wherein Hh/Smo signalling is utilized for inhibition of differentiation and for inhibition of apoptosis of tumor cells; accordingly another selective inhibitor of Hh/Smo signalling can be used in place of cyclopamine for the practice of treatment.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A, 1B, 1C, 1D: Rapid regressions of the cyclopamine-treated BCC's as indicated by disappeared tumor regions (exemplified by arrows), markedly decreased height from skin surface and by a loss of translucency in less than a week. 1A: BCC, located on left nasolabial fold, prior to treatment. 1B: Same BCC on the fifth day of topical cyclopamine treatment. 1C: BCC, located on forehead, prior to treatment. 1D: Same BCC on the sixth day of topical cyclopamine treatment.

FIG. 2A, 2B, 2C, 2D, 2E, 2F: Microscopic appearances of the cyclopamine- and placebo-treated BCC's, showing the cyclopamine-induced massive apoptotic death and removal of the tumor cells and the disappearance of tumor nodules to leave behind cystic spaces with no tumor cells. Skin areas corresponding to the pre-treatment positions of the BCC's were excised surgically on the fifth and sixth days of cyclopamine exposure with a margin of normal tissue and subjected to conventional fixation, sectioning and hermatoxylene-eosine staining for microscopic analyses. 2A: Large cyst in the dermis corresponding to the position of a disappeared tumor nodule showing no residual tumor cells. 2B: Similar cysts in another dermal area that contained BCC prior to, but not after, treatment with cyclopamine. 2C: Low power view of an area of the BCC shown on FIG. 1D showing residual cells and formation of a large cyst by the joining together of the numerous smaller cysts in between these cells. 2D: High power view from an interior region of the same residual BCC as in FIG. 2C showing greatly increased frequency of the apoptotic cells and the formation as well as enlargement of the cysts by the apoptotic removal of the BCC cells. 2E: High power view from a peripheral region of the same residual BCC as in FIG. 2C also showing greatly increased frequency of the apoptotic cells and the formation of cysts by the apoptotic removal of BCC cells. 2F: High power view from an internal area of a placebo-treated BCC showing typical neoplastic cells of this tumor and the absence of apoptosis. Original magnifications are 100× for 2A, 2B, 2C and 1000× for 2D, 2E, 2F.

FIG. 3A, 3B, 3C, 3D, 3E, 3F, 3G: Immunohistochemical analyses of the cyclopamine- and placebo-treated BCC's showing differentiation of all residual BCC cells under the influence of cyclopamine and the decrease of p53 expression in BCC's following exposure to cyclopamine. 3A and 3B: Absence of staining with the monoclonal antibody Ber-Ep4 in all residual cells of cyclopamine-treated BCC (3A) contrasted with the strong staining in placebo-treated BCC (3B) showing that all residual cells in the cycopamine-trated BCC's are differentiated to or beyond a step detected by Ber-Ep4. Ber-Ep4 is a known differentiation marker that stains the BCC cells as well as the undifferentiated cells of the normal epidermis basal layer and of hair follicles but not the differentiated upper layer cells of normal epidermis. 3C: Heterogenous labelling of the residual cells of a cyclopamine-treated BCC with the Ulex Europaeus lectin type 1 showing differentiation of some of the BCC cells all the way to the step detected by this lectin which normally does not label the BCC's or the basal layer cells of the normal epidermis but labels the differentiated upper layer cells. 3D and 3E: Decreased expression of p53 as detected by the monoclonal antibody DO-7 in cyclopamine-treated BCC's (3D) in comparison to the placebo-treated BCC's (3E). Expression of p53 is known to decrease upon differentiation of the epidermal basal cells and upon differentiation of cultured keratinocytes. It is also well known that the amount of of p53, detectable by DO-7, increases in cells when they are exposed to DNA-damaging agents. 3F and 3G: Consistent retraction of BCC's from stroma, which is a feature known to be associated with the arrest of tumor cell proliferation, seen in cyclopamine-treated (3F, arrow shows the retraction space) but not in placebo-treated (3G) tumors (difference of the cyclopamine- and placebo-treated BCC's in terms of retraction from stroma is seen also in 3D, 2C vs 3B, 3E). Original magnifications are 400× for 3A, 3B, 3D, 3E, 1000× for 3C and 100× for 3F, 3G. All immunohistochemical labellings are with peroxidase-conjugated streptavidin binding to biotinylated secondary antibody; labelling is indicated by the brown-coloured staining. Sections shown in 3F and 3G are stained with Periodic Acid-Schiff and Alcian blue.

FIG. 4A, 4B, 4C, 4D: Normal pattern of labelling of the cyclopamine-treated normal skin with Ber-Ep4 showing that the undifferentiated cells of normal epidermis and of hair follicles are preserved despite being exposed to the same schedule and doses of cyclopamine as the BCC's. 4A: Ber-Ep4 labelling of the basal layer cells of the epidermis treated with cyclopamine. 4B and 4C: Higher power views from different areas of cyclopamine-treated epidermis showing Ber-Ep4 labelling of the basal cells. 4D: High power view of a hair follicle treated with cyclopamine yet showing normal labelling with Ber-Ep4. Original magnification is 400× for 4A and 1000× for 4B, 4C, 4D. Immunohistochemical detection procedure is the same as in FIG. 3A, 3B; labelling is indicated by brown coloring.

FIG. 5A shows an ulcerated BCC in the upper nasal region of a 68-year old man prior to treatment.

FIG. 5B shows the same BCC as in FIG. 5A at the 54^{th} hour of cyclopamine application to its lower half.

FIG. 5C shows a section from the cyclopamine-applied half of the BCC at the 54^{th} hour. Hematoxylene-Eo sine (H&E) staining, 400× original magnification.

FIG. 5 D shows a section from the untreated region of the same BCC, H&E, 400× original magnification.

FIG. 5E shows a section from the cyclopamine applied half of the BCC at the 54^{th} hour with immunohistochemical staining for the Ki-67 antigen. 200× original magnification.

FIG. 5F shows a section from the untreated region of the same BCC with immunohistochemical staining for the Ki-67 antigen. 200× original magnification.

FIG. 6A shows a trichoepithelioma on the cheek of an 82-year old man prior to treatment.

FIG. 6B shows the same skin region as in FIG. 6A after 24 hours of treatment with cyclopamine.

FIG. 6C shows a section from the excised skin region shown in FIG. 6B with residual tumor cells. H&E, 400× original magnification.

FIG. 6D shows another area from the same tissue as in FIG. 6C. In addition to the numerous apoptotic cells and the formation of cystic structures by their removal, the tumor is seen to be infiltrated by mononuclear cells. H&E, 200× original magnification.

FIG. 7A shows a pigmented BCC in the lower eyelid of a 59-year old man prior to treatment.

FIG. 7B shows the same BCC as in FIG. 7A on the third day of treatment with cyclopamine.

FIG. 7C shows a section from the treated region of the BCC shown in FIG. 7B, H&E, 200× original magnification.

FIG. 7D shows a close up view of an area of residual tumor cells in a section from the treated region of the BCC shown in FIG. 7B, H&E, 400× original magnification.

FIG. 7E shows a section from a punch biopsy material obtained from the BCC shown in FIG. 7A prior to treatment, H&E, 400× original magnification.

FIG. 7F shows a section containing part of the BCC nodule marked by the arrow in FIG. 7A. Cyclopamine cream was not applied directly onto this nodule but cyclopamine could have diffused from the adjacent direct application area (left of the figure). The tissue was excised after 3 days of treatment and 6 days of non-treated follow-up. Immunohistochemical labelling with Ber-Ep4. Notice a gradient pattern of the Ber-Ep4 labelling in the direction of the diffusion of cyclopamine. 100× original magnification.

FIG. 8A shows photograph of a tumor grown into the lumen of trachea near tracheal bifurcation. Photograph was taken during bronchoscopic examination of the lung tumor and respiratory airways prior to the initiation of treatment.

FIG. 8B shows shows photograph of the same tumor as in FIG. 8A soon after the direct injection of medicament into it in the first session of medicament administrations. Slight bleeding from the tumor due to needle's insertion is seen.

FIG. 8C shows photograph of the same tumor as in FIGS. 8A and 8B on the fourth day of treatment. The photograph was taken before the start of the injections in the third session of treatment on day four. Marked decrease of size of the tumor relative to the pre-treatment size is seen. Normal tissues around the tumor exposed to the medicament do not show a sign of harming. A small hematoma in the shrinked tumor is visible.

### COLOR PRINTS

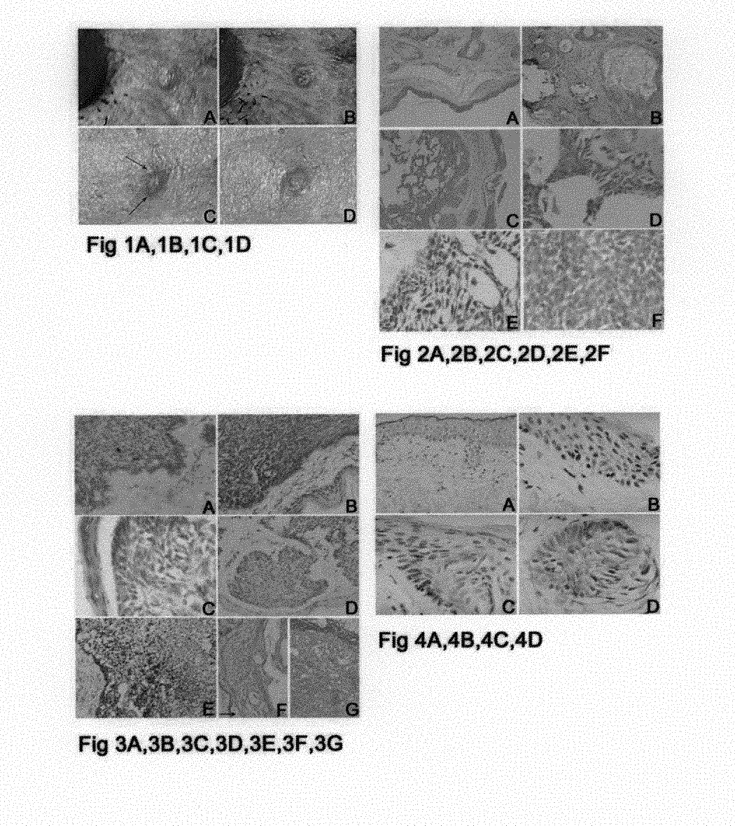

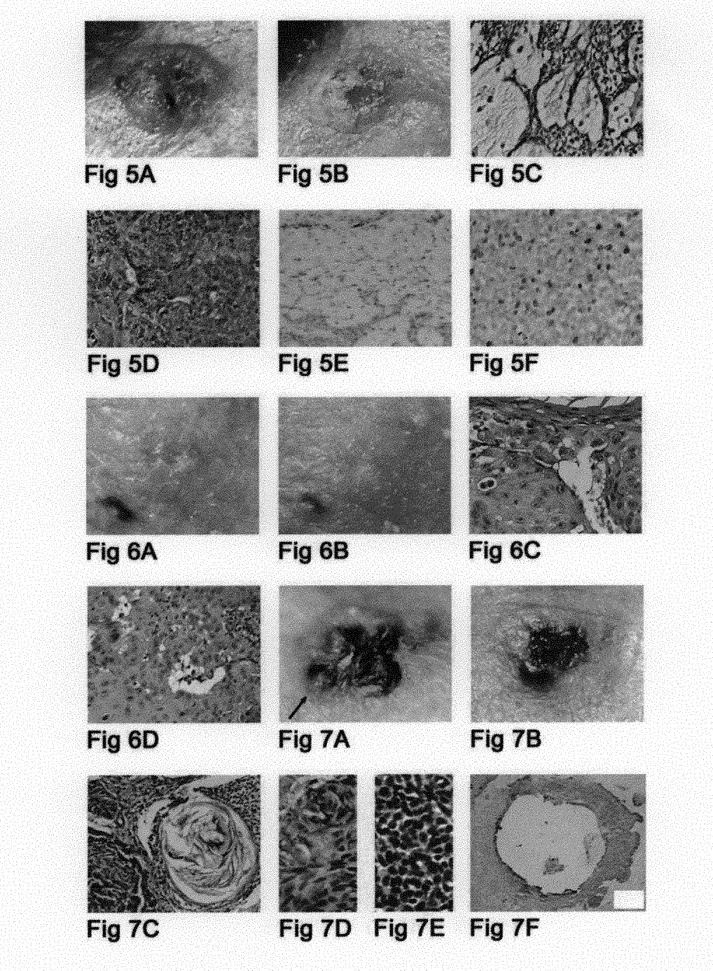

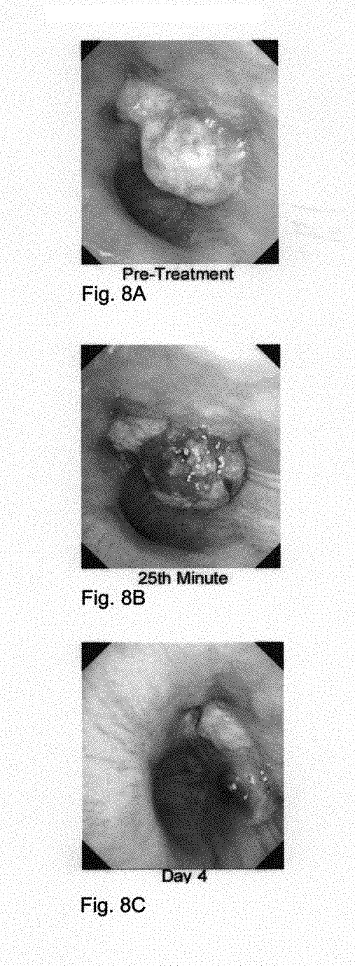

Color prints of the same figures as on pages 1/3 (FIG. 1A, 1B, 1C, 1D, FIG. 2A, 2B, 2C, 2D, 2E, 2F, FIG. 3A, 3B, 3C, 3D, 3E, 3G, FIG. 4A, 4B, 4C, 4D), 2/3 (FIG. 5A, 5B, 5C, 5D, 5E, FIG. 6A, 6B, 6C, 6D, FIG. 7A, 7B, 7C, 7D, 7D, 7E, 7F) and 3/3 (FIG. 8A, 8B, 8C), added as pages 1/3a, 2/3a and 3/3a, respectively, because the immunohistochemical data and findings, due to their nature, can be conveyed best in color rather than in grey-scale; we respectfully request consideration of this fact by the Patent Authority and the keeping of pages 1/3a, 2/3a and 3/3a as part of this patent application. However, pages 1/3a, 2/2a and 3/3a may be removed from the patent application if it is deemed necessary by the Patent Authority.

### DETAILED DESCRIPTION OF THE INVENTION

Cyclopamine was discovered as a teratogenic compound of Veratrum plants (Keeler R. F. (1969) Phytochemistry 8:223-225). It has been reported to inhibit differentiation of the precursors of the ventral cells in the developing brain (Incardona J. P. et al (1998) Development 125:3553-3562; Cooper M. K. et al. (1998) Science 280:1603-1607). Inhibition of cellular differentiation by cyclopamine has been reported in other systems as well, including the differentiation of bone marrow cells to erythroid cells (Detmer K. et al (2000) Dev. Biol. 222-242) and the differentiation of urogenital sinus to prostate (Berman D. M. et al (2000) J. Urol. 163-204). However, the opposite was found to be true in this invention with the tumor cells exposed to cyclopamine. Along with the cyclopamine-induced differentiation of tumor cells, apoptosis of tumor cells was also induced. Induction of tumor cell apoptosis by cyclopamine, again previously undescribed, is shown to be highly efficient. Furthermore, induction of apoptosis by cyclopamine was not secondary to a genotoxic effect and had extreme specificity; even the outer root sheath cells of hair follicles and normal epidermis basal cells that were adjacent to the tumor cells were well preserved while the tumor cells had differentiated and were undergoing apoptosis. Lack of adverse effects of the described treatment is confirmed also by the presence of clinically normal-looking healthy skin and hair at the sites of cyclopamine application in patients (longest duration of follow-up of a human subject is over 31 months at the time of writing and shows safety of the treatment also in the long term). Above summarised features of the treatment described in this invention make it highly desirable in cancer therapy and provide solutions to the long-standing problems of cancer therapy.

It is specifically contemplated that molecules can be derived from cyclopamine or synthesised in such a way that they possess structural features to exert similar receptor binding properties and biological/therapeutic effects as cyclopamine. Such a molecule is called here a “derivative of cyclopamine” and defined as follows: A molecule that contains the group of atoms of the cyclopamine molecule required for the binding of cyclopamine to its biological target but contains also modifications of the parent cyclopamine molecule in such ways that the newly derived molecule continues to be able to bind specifically to the same biological target to exert the biological effects of cyclopamine disclosed herein. Such modifications of cyclopamine may include one or more permissible replacement of or a deletion of a molecular group in the cyclopamine molecule or addition of a molecular group (particularly a small molecular group such as the methyl group) to the cyclopamine molecule, provided that the resultant molecule is stable and possesses the capability of specific binding to the same biological target as cyclopamine to exert the biological effects described herein. Derivation of such new molecules from cyclopamine can be readily achieved by those skilled in the art and the possession or lack of the biological effects of cyclopamine in the newly derived molecule can also be readily determined by those skilled in the art by testing for the biological effects disclosed herein.

For topical applications, cyclopamine can be dissolved in ethanol or another suitable solvent and mixed with a suitable base cream, ointment or gel. Cyclopamine may also be entrapped in hydrogels or in other pharmaceutical forms enabling controlled release and may be adsorbed onto dermal patches. In a pharmaceutical composition for topical administration, the cyclopamine or a salt or derivative thereof should be present in a concentration of 0.001 mM to 100 mM, preferably 12 to 24 mM. The effects shown in FIG. 1A to FIG. 1D, FIG. 2A to FIG. 2F, FIG. 3A to FIG. 3G and FIG. 4A to FIG. 4D have been obtained by a cream preparation obtained by mixing a solution of cyclopamine in ethanol with a base cream, so as to get a final concentration of 18 mM cyclopamine in cream. The base cream used is made predominantly of heavy paraffin oil (10% w/w), vaseline (10% w/w), stearyl alcohol (8% w/w), polyoxysteareth-40 (3% w/w) and water (68% w/w), but another suitably formulated base cream is also possible. Optimal concentration of cyclopamine in a pharmaceutical form as well as the optimal dosing and application schedules can obviously be affected by such factors as the particular pharmaceutical form, the localisation and characteristics of the skin containing the tumor (e.g. thickness of the epidermis) and the tumor size; however these can be determined by following well known published optimisation methods. The dosing and the application schedules followed for the tumors shown in FIG. 1A (BCC on the nasolabial fold, about 4×5 mm on surface) and FIG. 1C (BCC on the forehead, about 4×4 mm on surface) are as follows: 10±2 μl cream (containing 18 mM cyclopamine) applied directly onto the BCC's with the aid of a steel spatula four times per day, starting about 9.00 a.m. with about 3½ hours in between. Night-time applications, avoided in this schedule because of possible loss of cream from the patient skin to linens during sleep, can be performed by suitable dermal patches. Preservation of the undifferentiated cells in the normal epidermis and in hair follicles following exposure to cyclopamine, as described in this invention, provide information about the tolerable doses in other possible modes of administration as well; e.g. direct intratumoral injection of an aqueous solution or systemic administration of the same or of cyclopamine entrapped in liposomes.

FIG. 1A, FIG. 1B, FIG. 1C and FIG. 1D show rapid clinical regressions of the BCC's following exposure to cyclopamine. Besides the visual disappearance of several tumor areas within less than a week of cyclopamine exposure, there is a loss in the typically translucent appearance of the BCC's as seen by the comparison of FIG. 1B to FIG. 1A and of FIG. 1D to FIG. 1C.

FIG. 2A to FIG. 2F show microscopic appearances of the tumor areas subjected to surgical excisions together with a margin of normal tissue on the fifth and sixth days of cyclopamine applications when the BCC's had lost most of their pre-treatment areas but still possessed few regions that, although markedly decreased in height, had not yet completely disappeared and therefore had residual tumor cells for microscopic analyses.

FIG. 2A and FIG. 2B show, on tissue sections, the skin areas corresponding to the visually disappeared tumor nodules. The tumors are seen to have disappeared to leave behind large cystic structures containing little material inside and no detectable tumor cells.

FIG. 2C shows microscopic appearance of a skin area that contained still visible BCC in vivo. These regions are seen to contain residual BCC's displaying large cysts in the tumor center and smaller cystic structures of various sizes located among the residual BCC cells towards the periphery.

FIG. 2D and FIG. 2E show 1000× magnified appearances from the interior and palisading peripheral regions of these residual BCC's and show the presence of massive apoptotic activity among the residual BCC cells regardless of the tumor region. These high magnifications show greatly increased frequency of the BCC cells displaying apoptotic morphology and formation of the cystic structures by the apoptotic removal of cells, as exemplified in FIG. 2D by the imminent joining together of the three smaller cysts into a larger one upon removal of the apoptotic septal cells.

FIG. 2F shows that the BCC's treated with the placebo cream (i.e. the cream preparation identical to the cyclopamine cream except for the absence of cyclopamine in placebo) show, by contrast, the typical neoplastic BCC cells and no detectable apoptotic activity.

Cells undergoing apoptosis are known to be removed by macrophages and by nearby cells in normal tissues and the quantification of apoptotic activity by morphological criteria on hematoxylene-eosine stained sections is known to provide an underestimate. Despite these, the quantitative data shown in Table 1 show greatly increased apoptotic activity caused by cyclopamine among the residual BCC cells.

The loss of translucency in the cyclopamine-treated BCC's raises the intriguing possibility of differentiation of BCC's under the influence of cyclopamine. This possibility, which can be tested by immunohistochemical analyses of the BCC's, is found to be the case in this invention. In normal, epidermis, differentiation of basal layer cells to the upper layer cells is accompanied by a loss of labelling with the monoclonal antibody Ber-Ep4. Ber-Ep4 labels also the BCC cells and is a known marker for these neoplasms. FIG. 3A, FIG. 3B and the quantitative data on Table 1 show that, while Ber-Ep4 strongly labels all peripheral palisading cells and over 90% of the interior cells of the placebo-treated BCC's, none of the residual peripheral or interior cells of the cyclopamine-treated BCC's are labelled by Ber-Ep4. Differentiation of the BCC's under the influence of cyclopamine, hitherto unknown by any other means and highly unusual because of achievement of it in vivo and in all cells by immunohistochemical criteria, has independent value in the treatment of cancer.

Another differentiation marker, Ulex Europeaus lectin type 1, normally does not label the BCC's or the basal layer cells of normal epidermis but labels the differentiated upper layer cells. FIG. 3C, showing the heterogenous labelling of the residual cells of cyclopamine-treated BCC's with this lectin, shows differentiation of some of the BCC cells beyond the differentiation step detected by Ber-Ep4 all the way to the step detected by Ulex Europeaeus lectin type 1.

The p53 is a master regulator of the cellular response to DNA-damage. Amount of this protein is known to increase in the cell nucleus following exposure of cells to genotoxic agents. When the DNA-damage is increased beyond a threshold, p53 serves for the apoptotic death of cells. Radiation therapy of cancer and the genotoxic cancer chemotherapeutics that are currently common, act largely by this mechanism, i.e. by causation of apoptosis secondary to the damaging of DNA. The monoclonal antibody DO-7 can bind both normal and missense mutant (i.e. non-functional) forms of p53 and is known to be capable of detecting the increase of p53 in the cells following exposure to DNA-damaging agents.

FIG. 3D, FIG. 3E and the quantitative data in Table I show that both the DO-7 labelling intensity and the frequency of labelled cells are markedly decreased in cyclopamine-treated BCC's in comparison to the placebo-treated BCC's. Thus cyclopamine causes, not an increase, but rather a decrease of p53 in the nuclei of cyclopamine-treated BCC cells. Since expression of p53 is known to decrease in epidermal cells upon differentiation, the decreased DO-7 labelling of the cyclopamine-treated BCC's is likely to be secondary to the cyclopamine-induced differentiation of the BCC cells. In any case, massive apoptotic activity in the cyclopamine-treated BCC's despite markedly decreased p53 expression means that the cyclopamine-induced apoptosis of these tumor cells is by a non-genotoxic mechanism.

Arrest of the proliferation of BCC's is known to be associated with their retraction from stroma. Although retraction from stroma can also be caused artefactually by improper fixation and processing of the tissues, adherence to published technical details ensures avoidance of such artefacts. As shown in FIG. 3F and FIG. 3G, cyclopamine-treated, but not placebo-treated BCC's, are consistently retracted from stroma. Exposure of BCC's to cyclopamine thus appears to be associated also with an arrest of proliferation.

FIG. 4A to FIG. 4D show Ber-Ep4 labelling of the normal skin tissue found on and around the cyclopamine-treated BCC's. Different epidermal areas that were treated with cyclopamine are seen in FIG. 4A, FIG. 4B and FIG. 4C to display normal pattern of labelling with Ber-Ep4, i.e. labelling of the basal layer cells. Similarly, FIG. 4D shows normal Ber-Ep4 labelling of a hair follicle exposed to cyclopamine. Histological and immunohistochemical examinations of the cyclopamine-treated skin using antibodies to cytokeratin 15 and cytokeratin 19 (known to label the hair follicle outer root sheath cells with stem cell features) also revealed normal staining of hair follicles and revealed no adverse effect of the treatment on tissues and putative stem cells. Thus, the undifferentiated cells of normal epidermis and of hair follicles are preserved, despite being exposed to the same schedule and doses of cyclopamine as the BCC's. Further relevant in this regard is the display of normal skin and hair in the followed-up former treatment areas (as long as over 31 months at this writing) implying a lack of adverse effects also functionally.

Causation of highly efficient differentiation and apoptosis of the tumor cells in vivo by cyclopamine at doses that preserve the undifferentiated tissue cells are hitherto unknown achievements that, together with the non-genotoxic mode of action of cyclopamine, support the use of cyclopamine not only on BCC's but also on those internal tumors that utilize the hedgehog/smoothened pathway for proliferation and for prevention of apoptosis and/or differentiation.

FIG. 5A shows a large ulcerated BCC on the upper nasal region of a 68-year old man prior to treatment. Cyclopamine cream (18 mM in the base cream described above) was applied to the lower half of the BCC shown in FIG. 5A. Every third hour, about 20 μl cream was applied directly onto the lower half and the upper half was left untreated. Thus, the tumor cells in the uppermost part (FIG. 5A) are least likely to receive cyclopamine by possible diffusion form the directly applied region and will be exposed to relatively much lower concentrations of cyclopamine, if any. FIG. 5B shows the tumor on the 54^{th} hour of treatment just prior to surgical excision for investigation. While rapid regression of the tumor is evident in the cyclopamine-applied lower half, the region of the tumor furthest away from the directly applied half is seen to be relatively unaltered (FIG. 5B; the region towards the upper right corner of figure). FIG. 5C shows a hematoxylene-eosine stained section from the lower (cyclopamine-treated) part of the excised tissue. Numerous apoptic cells are seen together with variously sized cysts that form as a result of the death and removal of the tumor cells (FIG. 5C). In contrast, the non-treated region of the same tumor furthest away from the cyclopamine-applied half shows a solid tumor tissue with mitotic figures and no detectable apoptotic cells (FIG. 5D). FIG. 5E and FIG. 5F show the immunohistochemically stained tissue sections from the cyclopamine-treated and non-treated regions, respectively, of the tumor using the monoclonal antibody Ki-S5 (Dako A/S, Glostrup, Denmark) against the Ki-67 antigen. The Ki-67 antigen, which is a known marker of the proliferating cells, is no longer expressed in the cyclopamine-treated region of the tumor (FIG. 5E), while the tumor furthest away from the cyclopamine-applied region clearly display proliferative activity (FIG. 5F). Thus staining of the tissue sections with an antibody against the Ki-67 antigen shows again arrest of tumor cell proliferation by cylopamine under the conditions described.

Trichoepithelioma is another tumor associated with genetic changes that cause increased hedgehog-smoothened signalling (Vorechovsky L. et al. (1997) Cancer Res. 57:4677-4681; Nilsson M. et al. (2000) Proc. Natl. Acad. Sci. USA 97:3438-3443). FIG. 6A shows a trichoepithelioma on the cheek of an 82-year old man prior to treatment and FIG. 6B shows the same skin area after only 24 hours of exposure to the cyclopamine cream (18 mM cyclopamine in the base cream; about 25 μl cream was applied every third hour directly onto the tumor). Because of the rapid regression, treatment was discontinued on the 24^{th} hour and the entire skin area corresponding to the original tumor was excised for investigation. FIG. 6C and FIG. 6D show the tissue regions that contained residual tumor cells on the 24^{th} hour and reveal marked apoptotic activity among these residual tumor cells. Cystic spaces resulting from the apoptotic removal of tumor cells (FIG. 6C, FIG. 6D) as well as mononuclear cellular infiltration of tumor (FIG. 6D) are seen. Another noteworthy finding in this patient was the decreased size and pigmentation of a mole located nearby the treated tumor on the 24^{th} hour of treatment (FIG. 6B versus FIG. 6A). As cyclopamine could have diffused from the adjacent area of application, the mole (a benign melanocytic tumor) appears to be sensitive to relatively low concentrations of cyclopamine. Indeed, treatment of melanocytic nevi with the cyclopamine cream (18 mM cyclopamine in base cream) in another volunteer also caused similarly rapid depigmentation and disappearance of the nevi (data not shown). Thus, the invention is also suitable for cosmetic purposes, e.g. decreasing pigmentation in the hyperpigmented skin areas and lesions and improving the appearance of such skin areas.

FIG. 7A shows a pigmented BCC on the lower eyelid of a 59-year old man prior to treatment. Cyclopamine cream (18 mM cyclopamine in the base cream) was applied in this patient onto all of the nodules except for the one marked by the arrow. This nodule, which could have received cyclopamine only by diffusion from the adjacent treated region, would be exposed to a relatively lower concentration of cyclopamine. As the pigmented nature of this tumor facilitated clinical follow-up, treatment (application of about 20 cyclopamine cream, 18 mM cyclopamine in base cream, on every fourth hour) was discontinued on the third day when the tumor in the treated region had largely regressed but still contained visible parts (FIG. 7B). The tumor was then followed up without treatment for a study of the possible late effects. A clear further clinical regression was not observed in the absence of treatment and the skin area corresponding to the original tumor was excised on the sixth day of follow-up (ninth day from the start of treatment). Hematoxylene-eosine stained sections from the treated region of tumor revealed many cystic spaces that lacked tumor cells (FIG. 7C). The absence of an epithelium lining these cysts (FIG. 7C) is consistent with the representation by these cysts of the tissue areas that were formerly occupied by the tumor cells. At this time point (the sixth day of non-treated follow-up), tissue sections displayed a relative paucity of the apoptotic cells (FIG. 7C) consistent with the known rapidity of the clearance of apoptotic cells from live tissues. On the other hand, the residual tumor cells, particularly near the edges of cysts, showed unusually high frequencies of cells displaying features of spinous differentiation (e.g. the area towards the lower left of FIG. 7C; seen more clearly on higher magnification as exemplified from another area in FIG. 7D). Similar areas of differentiation or cysts were absent in the punch biopsy material obtained from the same tumor prior to the initiation of treatment (FIG. 7E). Other markers of differentiation also revealed induction of the differentiation of tumor cells by the treatment with cyclopamine. For example expression of the cell adhesion molecule CD44 is known to increase upon differentiation of the epidermal basal cells to the upper spinous layer cells (Kooy A J et al (1999) Human Pathology 30:1328-1335). We found weak, patchy and low frequency CD44 labelling in the punch biopsy material obtained from this BCC prior to the initiation of treatment and also in other untreated BCC's whereas the cyclopamine-treated BCC's exhibited markedly increased, strong labelling of essentially all residual tumor cells [labelling was with anti-human CD44 antibody F10-44-2 to the CD44 standard (Novocastra Labs Ltd, U.K.); data not shown].

The tumor nodule (marked by arrow in FIG. 7A) onto which we did not apply cyclopamine but could have received relatively lower concentrations by diffusion from the nearby application area, showed a large cystic center on the sixth day of follow-up (FIG. 7F). Immunohistochemical labelling of the sections through this nodule with Ber-Ep4 demonstrated a remarkable dose-response effect for the cyclopamine-induced differentiation of tumor cells (FIG. 7F; notice the absence of Ber-Ep4 labelling in the region of nodule towards the cyclopamine application area and the labelling in the region away from cyclopamine application). Importantly, it is also seen that the tumor cells that had differentiated beyond a critical step under the influence of cyclopamine (the Ber-Ep4 (−) cells on the side towards the area of cyclopamine application) had not reverted during the six days of non-treated follow-up. Thus while the tumor response to optimal concentrations of cyclopamine was rapid, suboptimal concentrations could not induce the differentiation (and apoptosis) of tumor cells.

FIG. 8A shows photograph of a tumor extending into the tracheal lumen in a man prior to treatment. Imaging of the patient by computed tomography, PET scanning and other imaging modalities showed a tumor occupying the superior and middle lobes and upper segment of inferior lobe of right lung. The tumor showed growths also to the outside of lung. Multiple lymph nodes in the mediastinum were involved. Right hilar region was nearly completely occupied and right pulmonary artery was surrounded by it. The tumor extended to the superior mediastinum and infracarinal regions and showed also signs of distant metastasis in whole body imaging. Histopathological investigations of bronchioalveolar lavage cells and of a biopsy obtained by bronchoscopy revealed adenosquamous carcinoma of lung. Patient had become severely dyspneic and bed-bound. Attending thoracic surgeon and physicians had concluded that surgical excision of tumor was not a therapeutic possibility and that the patient would also not benefit from radiotherapy and/or a drug treatment known in prior art. Patient's and his family's application for treatment by the instant treatment was evaluated and accepted. Repeats of the pathological and other laboratory and clinical examinations confirmed a malignant disease not treatable by a previously known treatment. Repeats of bronchoscopy and imaging revealed obstruction of the superior lobe's bronchus, involvements of other bronchi and narrowing of the tracheal lumen by the tumor. The photograph in FIG. 8A was taken near tracheal bifurcation during the bronchoscopic visualizations. The tumor of lung was estimated to have a volume of about 245 cubic centimeter by magnetic resonance imaging.

In view of the poor clinical status and weakness of patient and the severe dyspne associated with the obstruction and narrowing of the large airways, a two stage treatment strategy aiming to provide first adequate breathing and improvement of his general condition was decided. Under general anesthesia a medicament comprised of 18 mM cyclopamine in 98% ethanol, 2% phosphate buffered saline pH 7.4 was administered by direct injections into the tumoral growths into trachea and right lung's large airways with the aid of a bronchoscope. The medicament solution had been sterile filtered through a 0.2 μm pore size filter. A needle having 1.2 cm length was inserted to tumor to about 1 cm depth and about 2 ml of the medicament solution was administered during a period of about 5 minutes. The endoscopist was given latitude for injection around that rate of injection. Optimal number of the distances between each injection site varies depending on the configuration and size of a tumoral growth and optimal rate of injection can also vary depending on a particular tumor and interstitial fluid pressure in a tumor. Ideally an injection pump allowing accurate adjustment of rate of injection is preferred and an additional line joining the tubing near the needle and allowing co-administration of an appropriate diluent so that the concentration of ethanol exiting the needle can be reduced is preferred. Ethanol at high concentrations (e.g. absolute or 98%) is known to cause lysis of cell membranes to causes necrosis and to cause denaturation-precipitation of many proteins and direct injections of such concentrations of ethanol have long been used for causation of necrosis of small tumors by direct injection. Ethanol is readily miscible with aqueous media and can also help convection-enhanced delivery of drug molecules solubilized in it when intratumorally injected. A slow enough injection of a medicament solution having high concentrations of ethanol can also provide significant dilution of the small droplets of the ethanol carrier exiting the needle tip by the interstitial fluid in tumor. In the present example the tumoral growths into the airways were injected at positions about 2 to 3 cm apart under bronchoscopic visualization as above while slowly withdrawing the needle from the site of insertion and sterile saline administrations were used as needed, including for control of bleeding from a site of injection. In general the bleedings were minor and spontaneously ceased and instillation of cold saline was used for control of bleeding for a site showing continued bleeding. FIG. 8B shows photograph of the intratracheal portion of the tumor following intratumoral injection.

Medicament administrations with the aid of a bronchoscope can be repeated in multiple sessions under anesthesia. In this case about 12 to 18 ml of 18 mM cyclopamine solution was injected directly into tumor in a session as above and was also instilled to small airways along with saline. Following the first session, already through the end of the first day, the patient expressed ease of breathing. His physical examination and tests also showed improved respiration and lack of an adverse effect of the treatment. About 48 hours after the first session, the medicament administrations were repeated in a second session as above. The tumor sites injected in the first session were seen to show significant decrease of size relative to the pre-treatment size when visualized during the second session. On the fourth day after the first session, a third session of bronchoscopic visualization and medicament administrations were repeated as above. On the fourth day the tumor had become markedly reduced in size and the formerly obliterated right bronchus had opened. FIG. 8C shows the tumoral growth into trachea photographed on the fourth day at the start of the third session. It shows marked shrinkage of the tumoral growth into the tracheal lumen and the normal tissues around the tumor show no sign of an adverse effect.

The patient showed continued improvement of respiration and clinical status following the third session of medicament administrations. He was no longer bed bound and could walk and climb without help. A magnetic resonance imaging on the eighth day of the start of medicament administrations showed that the lung tumor had decreased to about 45% of the pre-treatment size and there were no signs of an adverse effect of the medicament administrations in mediastinal structures. Tumor shrinkage at distances several centimeters away from the about 1 cm inserted needle tip, the distances at which ethanol concentration would be reduced to 5% and less even if it would not be diluted by means other than a simple diffusion through that distance, showed that the therapeutic effect was due to the dose of the selective inhibitor of Hh/Smo signalling reaching there. Cyclopamine can associate with albumin, lipoproteins and other tissue molecules for movements in tissues. With these results and continued improvement of the clinical status of patient, objective of the first stage of his treatment was considered achieved for proceeding to the next stage of causation of tumor disappearance.

The dosing of a tumor patient according to the instant tumor treatment aims at apoptotic removal of tumor cells from the patient as described in this invention. It can be achieved while preserving normal tissue cells and functions of patient as described and exemplified. The Ber-EP4 labeled normal tissue cells, e.g. those in hair follicles, that are determined to be preserved following exposure to a medicament dose sufficing to induce differentiation and apoptosis of the tumor cells in the patient, are known to be relatively undifferentiated cells. In normal tissues the monoclonal antibody Ber-EP4 recognizes a protein synthesized by normal stem cells and multipotent progenitors and differentiation of these cells is accompanied by loss of expression of the protein that can be detected also by a number of other monoclonal antibodies generated against it (e.g. De Boer C J et al, Journal of Pathology 1999; 188:201-206; Kubuschok B et al, Journal of Clinical Oncology 1999; 17:19-24). Induction of apoptosis of tumor cells in a given patient by a dosing can be determined by one of various known methods. Histopathological examination of tumor cells for morphological signs of apoptosis and immunohistochemical and other methods of determining the molecular markers of apoptosing cells are known and can be used to determine whether or not a dose administered to a patient is sufficient for apoptotic removal of the tumor cells from the patient. Tumor cells can be obtained from a patient by conventional biopsying, aspiration with ultrasonic guidance of a catheter or by other known means depending on its site. Blood sampling from a vein can also be used to determine suitable molecular markers released to the extracellular fluid and thereby to blood plasma. In vivo imaging methods to visualize apoptosing cells are known and have the advantage of simultaneous visualization and measurement of tumor size. For example, in vivo imaging results using radiolabelled annexin V have been described to show significant positive correlation with the results of histopathological determination of apoptosing cells and uses of other molecular markers and additional methods of in vivo imaging of apoptosing cells are also known (e.g. D'Arceuil H et al, Stroke 2000; 32:2692-2700; Blankenberg F et al, Journal of Nuclear Medicine 2001; 42:309-316).

Liver and renal functions are generally involved in metabolism and excretions of drug molecules and it is known that other functions of a patient may also be needed to take into account in optimization of a dose of a medicament aiming to cause in him or her a previously known particular therapeutic effect. In case of a terminally ill cancer patient like in the lung cancer patient in the above example, a staged approach to improve first the general clinical condition of the patient and then to cause tumor disappearance can be followed. In the example of aforementioned lung cancer patient, following the first stage of treatment that improved his clinical status, systemic dosing was initiated to remove the tumor cells from the metastatic foci and tumor regions that extended from lung to mediastinal sites not suited for direct intratumoral injection. Non-oral systemic dosing was performed as cyclopamine is known to be acid-labile and it was the selective inhibitor of Hh/Smo signalling available for treating this patient at a cost that his family could meet.

Cyclopamine is a small hydrophobic molecule with little solubility in ordinary aqueous media. It can be solubilized in ethanol for preparation of a medicament for use in the present drug treatment. It can also be complexed with human albumin (obtained by methods of cloning of encoding sequences or by conventional methods) for preparation of a medicament for use in the treatment. Cyclopamine-albumin complex can be stored lyophilized and reconstituted to an aqueous solution before infusion to patient. Complexing of cyclopamine with a physiological macromolecule has the advantage of decreasing losses of pharmaceutically active molecule through glomerular filtration before reaching to the environs of the target tumor cells via systemic circulation. In the case of a medicament comprised of cyclopamine solubilized in ethanol for systemic administration, the rate of infusion should be adjusted by taking into account the actions of the ethanol carrier in patient. Ethanol normally forms in small amounts in every person. Amounts of the ethanol solvent to be administered for treatment of a patient having a metastatic tumor can however be large and toxicity by it must be avoided as follows. Ethanol is frequently consumed by adults for its sedating and other effects and patients can show variation in their ethanol metabolism (same mg/kg/day amount of ethanol administered to different persons can cause varying effects depending on e.g. whether a person is chronic alcohol drinker or non-drinker). In general up to about 11 mM blood ethanol concentration can be sedative, 11-33 mM can cause decrease or lack of motor coordination, 33-43 mM can cause reversible ethanol intoxication and blood concentrations more than about 70-80 mM can cause unconsciousness and ethanol can be fatal at still higher concentrations. Ethanol is however metabolized rapidly so that by adjusting the rate of infusion one can achieve adequate systemic dosing of a patient with cyclopamine solubilized in ethanol without causation of intolerable effects of ethanol in the patient. Blood ethanol concentrations can be monitored by known methods (including indirectly through measurements in breath) and typically what mg/kg ethanol administrations produce what blood concentrations are also known. The above mentioned ethanol effects can be used as a guide for not exceeding an ethanol concentration in blood that would be intolerable.

Various means of non-oral systemic administration of medicaments have been known. Infusion into a vein is frequently practiced and other means of non-oral systemic administration are also known (e.g. administration to peritoneal cavity with aid of a catheter for passage from there to the systemic circulation). Since ethanol at high concentrations (e.g. 98% or absolute ethanol) can cause lysis of plasma membrane of cells and precipitation of proteins, its rate of entry into a vein or peritoneal cavity must be slow enough to provide dilution to avoid such unwanted effects. Administration by use of a Y shaped catheter arrangement where one line provides the cyclopamine-ethanol solution, the other provides an aqueous diluting solution (e.g. saline) and the two are mixed just before entry into vein or peritoneal cavity can be practiced to dilute the ethanol concentration to about 5-10% (or lower). Rate of infusion of a solution form medicament containing cyclopamine (e.g. 18 mM cyclopamine in 98% ethanol) can be adjusted by taking into account the effects of the carrier as mentioned above. The dosing of tumor patient in the present treatment aims to cause apoptosis of the tumor cells while sparing normal cells and normal organ functions of the patient. It can be achieved as it has been described above and exemplified with patients having a tumor wherein Hh/Smo signalling is utilized for inhibition of differentiation and for inhibition of apoptosis of tumor cells. In the example of aforementioned lung cancer patient systemic infusion of a medicament comprised of 18 mM cyclopamine in 98% ethanol, 2% phosphate buffered saline pH 7.4 was performed as above by infusion during a period of about 8-10 hours to cause apoptotic removal of the tumor cells and tumor disappearance while preserving the normal cells and functions of patient. Calculations showed that these therapeutic effects were caused without exceeding 15 mg/kg/day cyclopamine dose in this case. Optimization of dosing of a patient takes into account his or her liver and kidney functions and other functions as it has been pointed. Induction of apoptosis of tumor cells can be monitored and tumor imaging can be performed as described above and tests of Hh/Smo signalling activity (e.g. expressions of one or more of patched 1, gli 1, gli 2, gli 3) in suitable cells from the patient (e.g. skin cells or others) can also be performed by known methods. Physical examination and observations of this lung cancer patient during and following administration did not reveal an intolerable adverse effect. Notably this patient was diagnosed to have coronary atherosclerosis and had undergone bypass operation and did not show a cardiovascular abnormality during and after non-oral systemic dosing that provided removal of his tumor cells by induction of apoptosis of them. Laboratory examinations of patient following such dosing also showed achievement of the therapeutic objective while preserving normal organ functions (Table 2).

Table 2 shows results agreeing with the clinical findings of patient that his normal organ functions, including those known to be ultimately depended on Hh/Smo signalling, were preserved while removing tumor cells from him by inducing their apoptosis. Alanine aminotransferase activity in blood serum is known to be a sensitive indicator of hepatocyte damage and increases after such damage. It was normal in the patient. Normal amylase activity is consistent with lack of damage in pancreas. Elevated lactate dehydrogenase activity would be consistent with the induction of apoptosis of tumor cells as this is an enzyme that is typically highly expressed in tumor cells. The slight elevation of bilirubin in blood serum involving mostly the direct bilirubin is interpreted to be due to the amount of the ethanol carrier administered. Normalcy of K⁺ concentration in blood serum and red blood cell indices are consistent with lack of erythrocyte lysis or other damage.

The efficiency of the described induction of apoptosis of tumor cells, while advantageous, is to be taken into consideration in treatment of patients. Uric acid is a metabolite that increases in blood plasma with increased catabolism of nucleic acids. Apoptosis of large numbers of tumor cells causes production of increased quantities of uric acid. Elevation of uric acid in blood plasma can be managed by attending physicians of patient by use of allopurinol and also by fluid loading (e.g. with saline) to enhance excretion of it. The elevated blood serum uric acid in the above lung cancer patient (Table 2) is again consistent with the efficient apoptotic removal of tumor cells from patient by the instant treatment.

Pharmaceutically acceptable drug molecules that provide selective inhibition of Hh/Smo signalling can be made and used in place of cyclopamine for practice of the instant tumor treatment of patients having a tumor where Hh/Smo signalling is utilized for inhibition of differentiation and for inhibition of apoptosis of tumor cells. Such a drug molecule can be derived from cyclopamine without a priori restriction of structural features as long as the derivative performs the function of cyclopamine. Cyclopamine is known to be a selective inhibitor of Hh/Smo signalling and the above pointed nature of the target tumors of instant treatment also calls for use of a pharmaceutically acceptable molecule that provides selective inhibition of Hh/Smo signalling for the described treatment. Molecules that provide selective inhibition of Hh/Smo signalling and having no structural relation to cyclopamine are known and can be newly identified by use of known screening methods (e.g. Sasaki H et al, Development 1997; 124; 1313-1322) and testing of positives in a known animal model (e.g. Ericson J et al, Cell 1996; 87:661-673; Incardona J P et al, Development 1998; 125:3553-3562; Stenkamp D L et al, Developmental Biology 2000; 220:238-252; Nasevicius A et al, Nature Genetics 2000; 26:216-220).

These examples illustrate effectiveness of the described treatment in the causations of tumor cell differentiation and apoptosis and in obtaining rapid clinical regression of the tumors displaying hedgehog/smoothened signalling. Effectiveness on several independent tumors in unrelated patients with differing genotypes is consistent with the general utility of the described treatment.

Of the numerous substances known in the art to display inhibitory activity on tumor cell proliferation, only a small minority prove to be usable or effective in the treatment of tumors in patients. A major reason for this is the causation of harm also to the normal cells (particularly to the progenitor and stem cells) and the development of intolerable adverse effects. As hedgehog/smoothened signalling is well known to be employed by several normal cell types and for the maintenance of stem cells (Zhang Y et al (2001) Nature 410:599-604), use of cyclopamine on tumors of patients would have been anticipated to lead to adverse effects, especially on the normal tissues around tumors that are exposed to the same schedule and doses of cyclopamine as the tumors. However, treatment with cyclopamine under the described conditions has not revealed undue adverse effects on normal tissue components (including the putative stem cells) by histological/immunohistochemical criteria. Moreover, former skin sites of cyclopamine application that have been followed up more than 31 months at the time of this writing continue to display healthy-looking normal skin and hair, suggesting functional preservation as well of the stem cells and long-term safety. Our finding that a transient exposure to cyclopamine can suffice for the causations of tumor cell differentiation and apoptosis is further surprising and facilitates treatment of internal tumors as well. The term transient administration of cyclopamine for treatment as used here means administration of cyclopamine for a period that is short enough so that causation of the apoptosis and/or differentiation of the normal tissue cells do not happen to such an extend to lead to intolerable adverse effects. We describe in this invention that tumor cells can be caused to undergo apoptosis and/or differentiation in vivo much faster than normal tissue cells so that during the same period of exposure to cyclopamine relatively much smaller proportion or no normal tissue cells undergo cyclopamine-induced apoptosis and/or differentiation, making thereby the clinically detectable or intolerable adverse effects minimal or nonexistent. It is also clear that the therapeutic effectiveness described herein and the rapid disappearance of treated tumors could not be possible without the causation of tumor cell apoptosis since merely inhibiting or slowing the tumor cell proliferation by cyclopamine would, at best, help one only to keep the tumor at its pre-treatment size.

TABLE 1
Induction of the Differentiation and Apoptosis of Basal Cell Carcinoma
Cells by Topical Cyclopamine

| | Peripheral | | Non-Palisading | |
|---|---|---|---|---|
| | Palisading Cells | | Cells of | |
| | of the BCC's | | the BCC's | |
| | Treated with | | Treated with | |
| | Placebo | Cyclopamine | Placebo | Cyclopamine |
| % of Cells | 0 ± 0 | 20 ± 8 | 0.2 ± 0.4 | 18 ± 11 |
| showing ≧2 | | | | |
| Morphological | | | | |
| Signs of Apoptosis | | | | |
| on H&E Stained | | | | |
| Tissue Sections | | | | |
| % of Cells Labelled | 100 ± 0 | 0 ± 0 | 91 ± 8 | 0 ± 0 |
| with Ber-Ep4 | | | | |
| % of Cells Labelled | 58 ± 27 | 16 ± 11 | 67 ± 22 | 5 ± 3 |
| with DO-7 | | | | |

Means ± standard deviations from at least 16 randomly selected high-power (1000 X) fields of the tissue sections of each tumor group are shown.
p < 0.001 for the placebo vs. cyclopamine-treated tumors for all the parameters, both for the palisading peripheral and the non-palisading (interior) tumor areas.

TABLE 2
Examples Of Clinical laboratory Test Results Showing
Preservation Of The Normal Cells and Normal Organ
Functions Of Patient Following Systemic Dosing With
A Medicament Comprised Of A Selective Inhibitor Of
Hedgehog/Smoothened Signaling

| | Result Of | | |
|---|---|---|---|
| Analyte | Measurement In Patient | | Referans Range |
| Alanine aminotransferase | 35 | IU/L | 5-41 |
| Amylase | 30 | IU/L | <90 |
| Aspartate aminotransferase | 47 | IU/L | 6-38 |
| Lactate dehydrogenase | 1070 | IU/L | 240-480 |
| Uric acid | 13.2 | mg/dL | 3.4-7.0 |
| Total bilirubin | 2.33 | mg/dL | <1.1 |
| Direct bilirubin | 1.57 | mg/dL | <0.3 |
| K⁺ | 4.59 | mM | 3.5-5.5 |
| Erythrocyte count | 4.43 × 10⁶/ | μL | 4.00-5.80 |
| Hemoglobin | 11.7 | g/dL | 12.0-17.5 |
| White blood cell count | 11.5 × 10³/ | μL | 4.5-11.0 |

Blood samples of the lung cancer patient in the exemplification were analysed following non-oral systemic dosing of the patient with a medicament comprised of cyclopamine (18 mM) in 98% ethanol, 2% phosphate buffered saline pH 7.4.

1. A method for treatment of a human subject having a tumor, comprising
determining that the tumor in the subject is a tumor wherein Hedgehog/Smoothened signaling is utilized for inhibition of apoptosis of tumor cells, and
administering to the subject a medicament comprised of a pharmaceutically acceptable molecule that selectively inhibits Hedgehog/Smoothened signaling,
wherein said medicament is administered in a dosing that is sufficient to cause apoptosis of said tumor cells and decrease of size or disappearance of the tumor and the subject continues to have normal tissue cells showing labeling with the monoclonal antibody Ber-EP4.

2. A method according to claim 1, wherein said molecule is cyclopamine or a functionally equivalent derivative of cyclopamine.

3. A method according to claim 1, wherein apoptosis of tumor cells and decrease of size or disappearance of the tumor in the subject are caused without genotoxicity.

4. A method according to claim 1, wherein said medicament is formulated for topical or systemic administration or for intratumoral injection or is adsorbed onto a dermal patch or is a controlled release or liposomal formulation or is in the form of a cream or ointment or gel or hydrogel.

5. A method for treatment of a human subject having a tumor, comprising
determining that the tumor in the subject is a tumor wherein Hedgehog/Smoothened signaling is utilized for inhibition of differentiation and for inhibition of apoptosis of tumor cells, and
administering to the subject a medicament comprised of a pharmaceutically acceptable molecule that selectively inhibits Hedgehog/Smoothened signaling,
wherein said medicament is administered in a dosing that is sufficient to cause differentiation and apoptosis of said tumor cells and decrease of size or disappearance of the tumor and the subject continues to have normal tissue cells showing labeling with the monoclonal antibody Ber-EP4.

6. A method according to claim 5, wherein said molecule is cyclopamine or a functionally equivalent derivative of cyclopamine.

7. A method according to claim 5, wherein apoptosis of tumor cells and decrease of size or disappearance of the tumor in the subject are caused without genotoxicity.

8. A method according to claim 5, wherein said medicament is formulated for topical or systemic administration or for intratumoral injection or is adsorbed onto a dermal patch or is a controlled release or liposomal formulation or is in the form of a cream or ointment or gel or hydrogel.

9. A method for treatment of a human subject having a tumor, comprising
determining that the tumor in the subject is a tumor wherein Hedgehog/Smoothened signaling is utilized for inhibition of apoptosis of tumor cells, and
administering to the subject a medicament comprised of cyclopamine or another pharmaceutically acceptable molecule that like cyclopamine selectively inhibits Hedgehog/Smoothened signaling,
wherein said medicament is administered in a dosing that is sufficient to cause apoptosis of said tumor cells and decrease of size or disappearance of the tumor and the subject continues to have normal tissue cells showing labeling with the monoclonal antibody Ber-EP4.

10. A method according to claim 9, wherein said another molecule is a functionally equivalent derivative of cyclopamine.

11. A method according to claim 9, wherein apoptosis of tumor cells and decrease of size or disappearance of the tumor in the subject are caused without genotoxicity.

12. A method according to claim 9, wherein said medicament is formulated for topical or systemic administration or for intratumoral injection or is adsorbed onto a dermal patch or is a controlled release or liposomal formulation or is in the form of a cream or ointment or gcl or hydrogel.

13. A medicament for treatment of a human subject having a tumor wherein Hedgehog/Smoothened signaling is utilized for inhibition of apoptosis of tumor cells,
comprising a pharmaceutically acceptable molecule that selectively inhibits Hedgehog/Smoothened signaling,
wherein said medicament is administered in a dosing that is sufficient to cause apoptosis of said tumor cells and decrease of size or disappearance of the tumor and the subject continues to have normal tissue cells showing labeling with the monoclonal antibody Ber-EP4.

14. A medicament according to claim 13, wherein said molecule is cyclopamine or a functionally equivalent derivative of cyclopamine.

15. A medicament according to claim 13, wherein said medicament is formulated for topical or systemic administration or for intratumoral injection or is adsorbed onto a dermal patch or is a controlled release or liposomal formulation or is in the form of a cream or ointment or gel or hydrogel.
